(19) United States
(12) Patent Application Publication
(10) Pub. No.: US 2025/0241622 A1
(43) Pub. Date: Jul. 31, 2025

(54) METHODS AND SYSTEMS FOR GENERATING DYNAMIC 3D ULTRASOUND IMAGE

(71) Applicant: GE Precision Healthcare LLC, Waukesha, WI (US)

(72) Inventors: Carmit Shiran, Middleton, WI (US); Doron Shaked, Haifa (IL); Lev Greenberg, Haifa (IL); Alexander Sokulin, Haifa (IL); Radhika Madhavan, Niskayuna, NY (US); Menachem Halmann, Monona, WI (US)

(21) Appl. No.: 18/427,685

(22) Filed: Jan. 30, 2024

(51) Int. Cl. A61B 8/08; A61B 8/00

(52) U.S. Cl. CPC A61B 8/483; A61B 8/0875; A61B 8/4245; A61B 8/4444; A61B 8/4494; A61B 8/466; A61B 8/5207; A61B 8/5269

(57) ABSTRACT

Various methods and systems are provided for a medical imaging system. In one embodiment, a method for generating a dynamic, three-dimensional (3D) ultrasound image comprises acquiring dynamic 3D ultrasound image data, generating a plurality of 3D rib space segments from the dynamic 3D ultrasound data, generating a dynamic panoramic 3D ultrasound image depicting the plurality of dynamic 3D rib space segments in an anatomical order, temporally synchronizing the plurality of dynamic 3D rib space segments, and outputting the dynamic panoramic 3D ultrasound image for display.

### FIELD

Embodiments of the subject matter disclosed herein relate to ultrasound imaging and, in particular, to visualizing rib spaces of a subject as a dynamic three-dimensional (3D) image.

### BACKGROUND

An ultrasound imaging system typically includes an ultrasound probe that is applied to a patient's body, and a workstation or device that is operably coupled to the ultrasound probe. During a scan, the ultrasound probe may be controlled by an operator of the system and is configured to transmit and receive ultrasound signals that are processed into an ultrasound image by the workstation or device. The workstation or device may show the ultrasound images as well as a plurality of user-selectable inputs through a display device. The operator or other user may interact with the workstation or device to analyze the images displayed on and/or selected from the plurality of user-selectable inputs.

As one example, ultrasound imaging may be used for examining a patient's lungs due to an case of use of the ultrasound imaging system at a point-of-care and resource availability relative to a chest x-ray or a chest computed tomography (CT) scan, for example. Further, the ultrasound imaging system does not expose the patient to radiation. Lung ultrasound imaging, also termed lung sonography, includes interpreting topography of a lung pleura for diagnostic purposes. Ultrasound image data captured by the ultrasound imaging system may be static or dynamic image data, and may be two-dimensional (2D) or three-dimensional (3D) image data.

### BRIEF DESCRIPTION

This summary introduces concepts that are described in more detail in the detailed description. It should not be used to identify essential features of the claimed subject matter, nor to limit the scope of the claimed subject matter. In one aspect, a method for generating a dynamic three-dimensional ultrasound image comprises acquiring dynamic three-dimensional ultrasound image data, generating a plurality of three-dimensional rib space segments from the dynamic three-dimensional ultrasound data, generating a dynamic three-dimensional ultrasound image depicting the plurality of three-dimensional rib space segments in an anatomical order, temporally synchronizing the plurality of dynamic three-dimensional rib space segments, and outputting the dynamic three-dimensional ultrasound image for display.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present disclosure will be better understood from reading the following description of non-limiting embodiments, with reference to the attached drawings, wherein below:

FIG. 1 shows a block schematic diagram of an ultrasound imaging system, according to an embodiment;

FIG. 2 is a schematic diagram illustrating an image processing system for processing three-dimensional (3D) ultrasound image data, according to an embodiment;

FIG. 3 shows an ultrasound image probe of an ultrasound imaging system, such as the ultrasound imaging system of FIG. 1;

FIG. 4 illustrates a thoracic cavity of an imaging subject, according to an embodiment;

FIG. 5 shows a high level flowchart of a method for generating a dynamic 3D ultrasound image, according to an embodiment;

FIG. 6 shows a flowchart of a method for generating a dynamic 3D ultrasound image, according to an embodiment;

FIG. 7 shows an example annotated 3D rib space segment that may be used to generate a 3D pleural surface, according to an embodiment;

FIG. 8 shows a first example dynamic panoramic 3D ultrasound image, according to an embodiment;

FIG. 9 shows a second example dynamic 3D ultrasound image, according to an embodiment; and

FIG. 10 shows an example display including a dynamic 3D ultrasound image, according to an embodiment.

### DETAILED DESCRIPTION

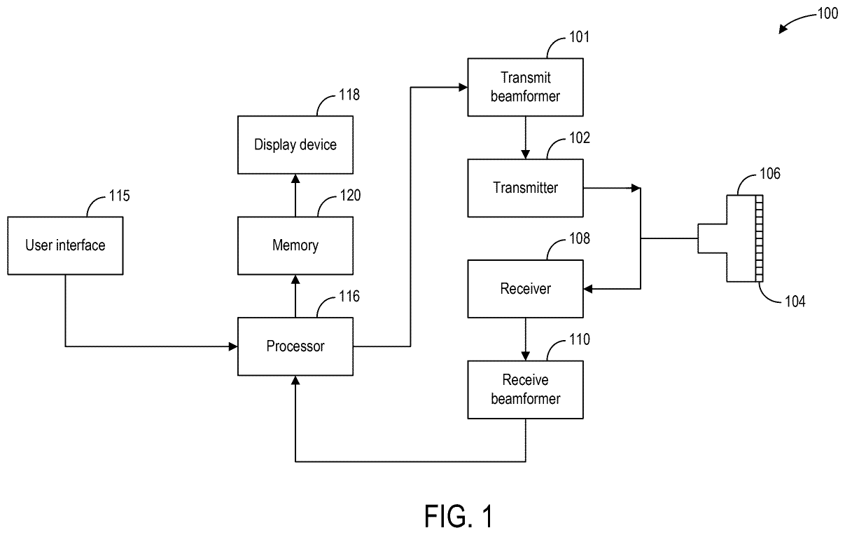

Embodiments of the present disclosure will now be described, by way of example, with reference to the FIGS. 1-10, which relate to various embodiments for generating a dynamic three-dimensional ultrasound image from 3D ultrasound image data. An ultrasound probe of an ultrasound imaging system may be configured to capture static and/or dynamic images, where dynamic images include a plurality of image frames acquired at different points in time. Displaying ultrasound image data as dynamic images allows visualization of how an imaging subject's anatomy changes over time. A size of a field-of-view (FOV) of the ultrasound probe dictates a maximum area (e.g., in 2D ultrasound image data) and/or a maximum volume (e.g., in 3D ultrasound image data) that may be acquired in a single frame of the ultrasound image data. A single frame of the ultrasound image data may therefore not capture one or more items of interest due to the size of the FOV, especially when a large organ and/or a large region of the imaging subject's body is being imaged. In order to minimize a likelihood of missing one or more items of interest in the ultrasound image data, particularly when imaging organs that are relatively large in comparison to the FOV, some conventional ultrasound imaging systems are configured to generate panoramic views. For example, a FOV of an ultrasound probe used to capture image data of a thoracic cavity of a patient may be sized to capture one to two rib spaces at a time. A panoramic view may be generated from multiple ultrasound images acquired at different spatial locations. The ultrasound images may be combined or stitched together in order to provide a single panoramic view that covers a larger area or volume than is covered in a single ultrasound image, due to constraints imposed by the size of the FOV.

Additionally, the ultrasound imaging system may capture two-dimensional (2D) and/or three-dimensional (3D) ultrasound image data. For example, the ultrasound probe of the ultrasound imaging system may be configured to acquire dynamic 3D ultrasound image data. Some conventional methods for displaying 3D ultrasound images from 2D ultrasound image data may be processing intensive, as the 2D ultrasound image data may be processed and modified to generate a 3D image from a stack of 2D ultrasound image data. In another example, a 3D mesh may be generated from the 2D ultrasound image data, and a 3D image may be generated from the 3D mesh. Thus, when a 3D ultrasound image is desired, it may be advantageous to use an ultrasound probe capable of capturing 3D ultrasound image data to perform an imaging scan.

In imaging scans where dynamic 3D ultrasound image data is acquired, there may be challenges in displaying items or regions of interest captured in the dynamic 3D ultrasound image data. For example, dynamic 3D ultrasound image data may be acquired continuously (e.g., as an uninterrupted sequence of image frames) or as multiple separate sequences of multiple image frames (e.g., where each of the multiple sequences may have the same or a different number of image frames). As described above, a size of an ultrasound probe FOV may restrict an area or volume of the imaging subject that is captured in a single image. Thus, multiple dynamic 3D ultrasound images may be captured when moving the ultrasound probe across the external surface of the imaging subject to capture image data of all of the regions of interest. In conventional ultrasound imaging methods, each of the multiple dynamic 3D ultrasound images may be stored and displayed as individual images, and a user may navigate among and select one or more images to be displayed and viewed for analysis and diagnosis. Additionally, the dynamic 3D ultrasound image data may show regions of interest of the imaging subject in an order that is different from a true anatomical order, and/or may show regions of interest for different durations of time. Segments of the dynamic 3D ultrasound image data that show different regions of interest may therefore have differences among various imaging subject vitals, such as respiratory cycle or heartrate. It is known to temporally synchronize and temporally scale dynamic 2D images (e.g., videos) based on a parameter such as respiratory cycle or heartrate in a panoramic view, such that each video in a panoramic view takes the same amount of time to play. However, this method may not be sufficient for displaying dynamic 3D images in a panoramic view that is true to imaging subject anatomy.

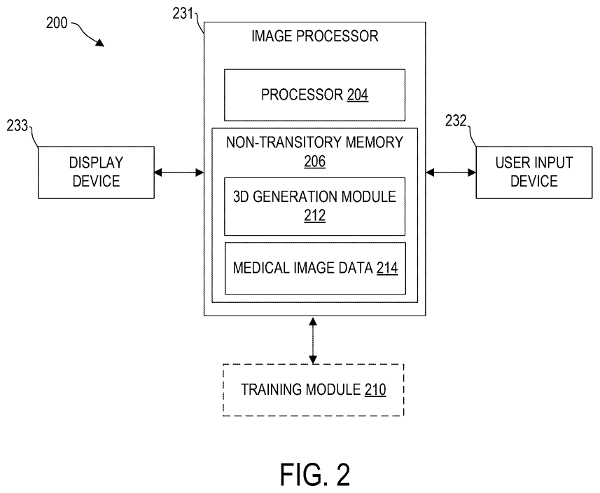

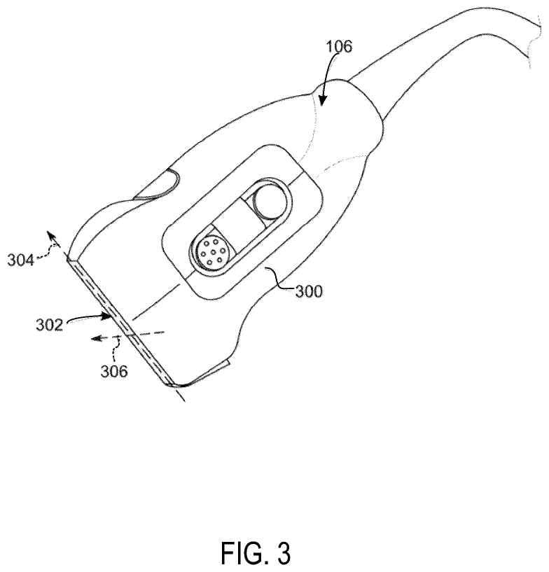

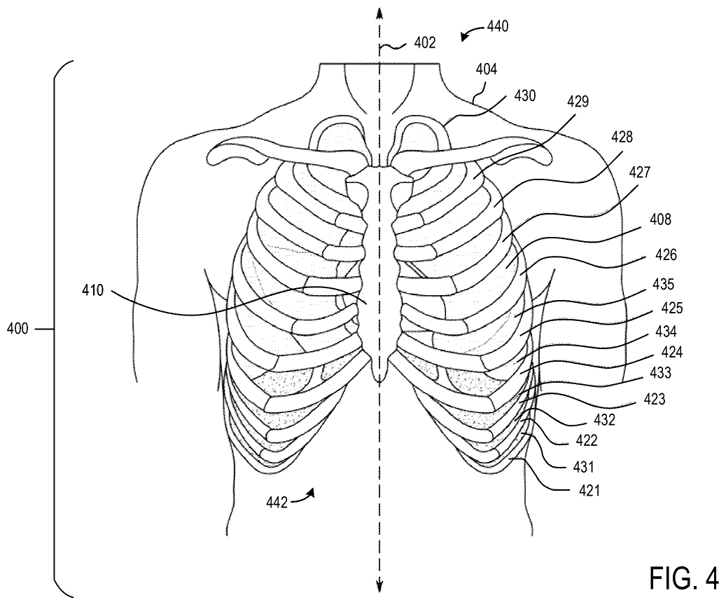

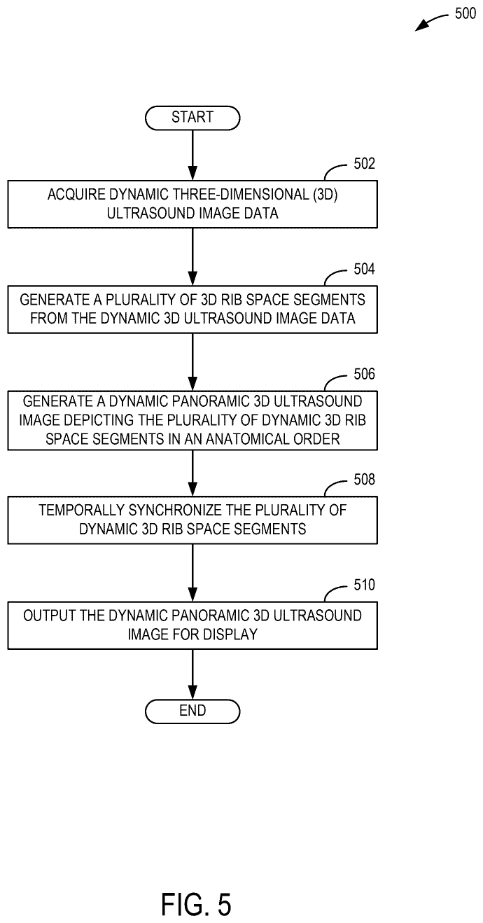

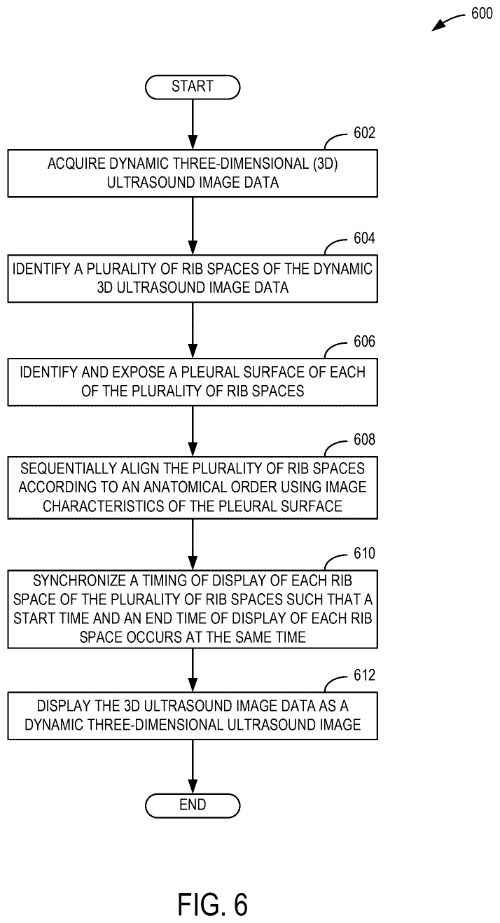

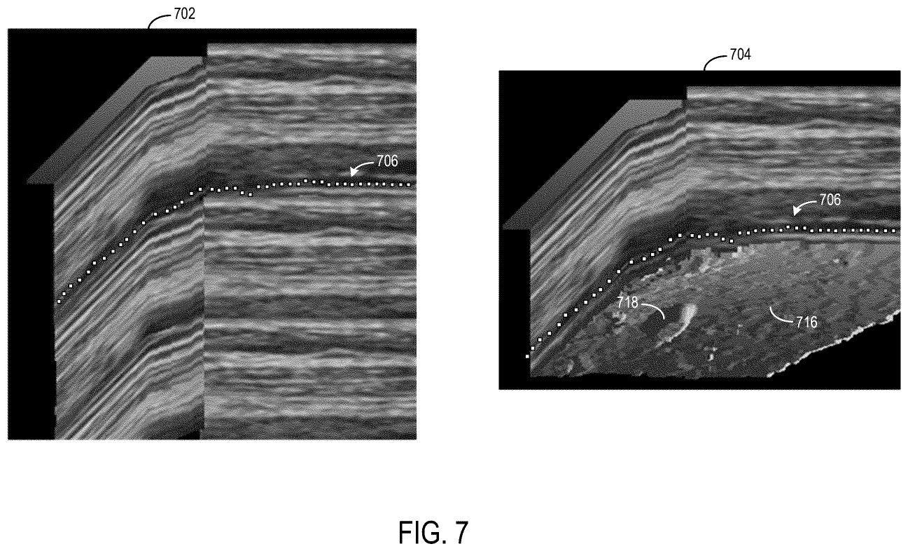

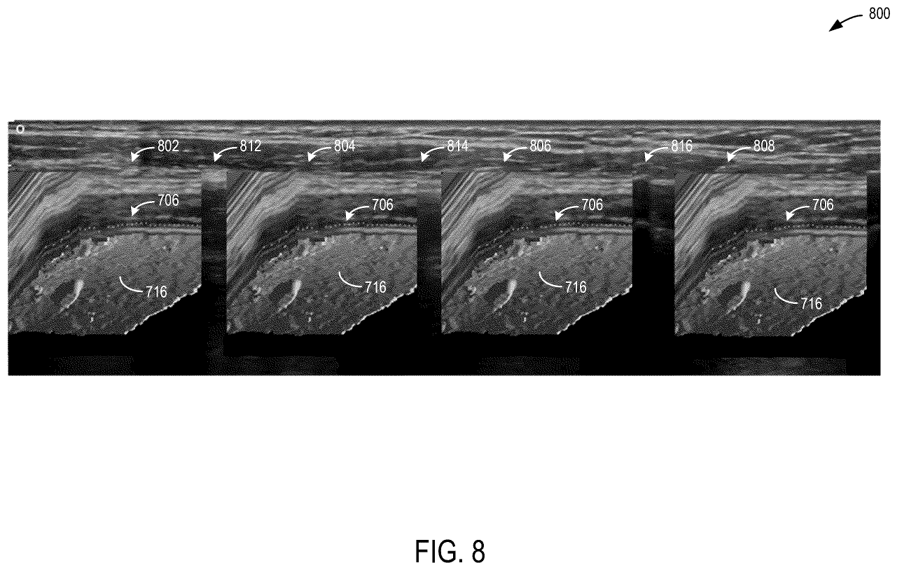

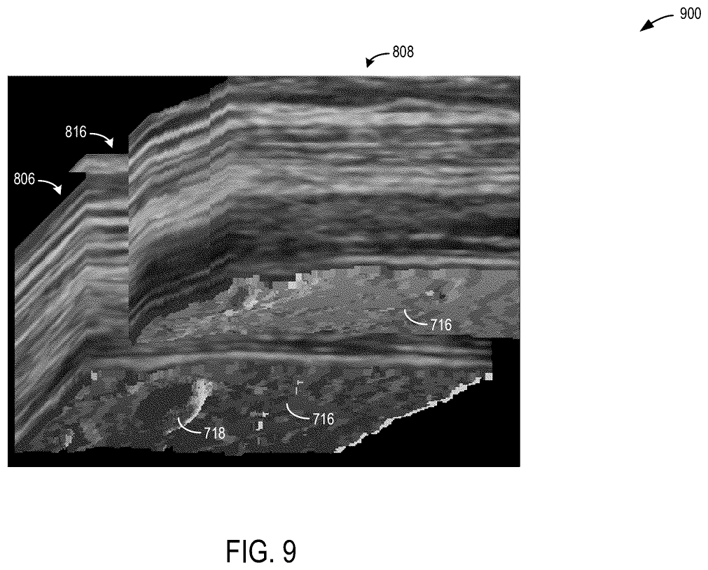

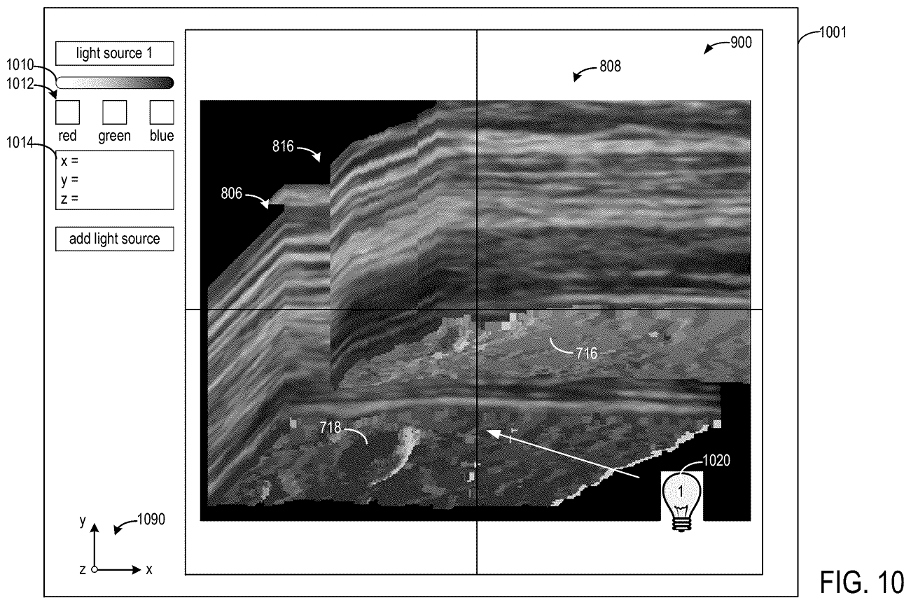

A dynamic 3D ultrasound image may be generated from dynamic 3D ultrasound image data captured by an imaging system, such as the ultrasound imaging system shown in FIG. 1. As the processes described herein may be applied to pre-processed imaging data and/or to processed images, the term “image” is generally used throughout the disclosure to denote both pre-processed and partially-processed image data (e.g., pre-beamformed RF or I/Q data, pre-scan converted RF data), as well as fully processed images (e.g., scan converted and filtered images ready for display). An example image processing system that may be used to generate the dynamic 3D ultrasound image is shown in FIG. 2. The ultrasound imaging system of FIG. 1 includes an ultrasound probe, an example of which is shown in FIG. 3. An imaging scan used to capture 3D ultrasound image data is performed by moving the ultrasound probe over an external surface of an imaging subject, such as a patient. Described herein are methods for generating a dynamic 3D ultrasound image of rib spaces, interior lung surfaces, and other structures of a thoracic cavity of a patient, an example of which is shown in FIG. 4. FIG. 5 shows a flow chart of a high-level method for generating the dynamic 3D ultrasound image from 3D ultrasound image data. FIG. 6 shows a flow chart of a more detailed method for generating the dynamic 3D ultrasound image from dynamic 3D ultrasound image data, including generating a plurality of 3D rib space segments from the dynamic 3D ultrasound image data, identifying and exposing a pleural surface of each of the 3D rib space segments, and ordering the plurality of 3D rib space segments in an anatomical order. The plurality of 3D rib space segments may be generated using rib shadows, and ordered in the anatomical order using pleural surface characteristics of each 3D rib space segment. FIG. 7 shows an example annotated 3D rib space segment that may be used to generate the dynamic 3D ultrasound image. FIG. 8 shows a first example dynamic 3D ultrasound image, shown in a panoramic view. FIG. 9 shows a second example dynamic 3D ultrasound image, shown in a perspective view. FIG. 10 shows an example display, for example of the ultrasound imaging system of FIG. 1, including a view of the second example dynamic 3D ultrasound image of FIG. 9. In this way, 3D images of multiple rib space segments and other thoracic cavity ultrasound images may be visualized in a single, dynamic image.

Displaying data of interest (e.g., displaying 3D images of multiple rib space segments with exposed pleural surfaces) in a single, dynamic 3D ultrasound image allows the user to see the most relevant data without having to navigate among, select, and analyze multiple ultrasound images. The speed of a user's navigation through various views (e.g., various rib space segments) may be increased because the methods described herein for guided display (e.g., navigation) saves the user from selecting an image of a rib space segment from an array of all images of the 3D ultrasound image data that may be displayed simultaneously or individually. In conventional methods, identifying a region of interest (e.g., a rib space of the 3D ultrasound image data potentially having abnormalities which may be indicative of a disease state) may include enlarging and/or shrinking one or more ultrasound images multiple times to compare ultrasound images of the set of 3D ultrasound image data. Additionally, as the 3D ultrasound image data is dynamic (e.g., comprised of multiple frames), conventional display and analysis methods may demand that a user further navigate among a duration of a single dynamic ultrasound image to view detail of the 3D ultrasound image. Further, the user may navigate among multiple dynamic ultrasound images (e.g., showing different regions of interest) and durations thereof to compare potential abnormalities among the multiple dynamic ultrasound images. This may pose further challenges, as each of the multiple dynamic ultrasound images may have different durations. Rather than stepping through selection, enlargement, and analysis of multiple 3D ultrasound images to identify relevant medical information, using the method described herein, the user may view the display of the display device, which has a single, dynamic 3D ultrasound image displayed thereon that includes dynamic 3D ultrasound data captured from multiple regions of the imaging subject (e.g., which may not be able to be captured simultaneously by the FOV of the ultrasound probe) arranged according to an anatomical order.

Referring to FIG. 1, a schematic diagram of an ultrasound imaging system 100 in accordance with an embodiment of the disclosure is shown. However, it may be understood that embodiments set forth herein may be implemented using other types of medical imaging modalities (e.g., magnetic resonance imaging, computed tomography, positron emission tomography, and so on). The ultrasound imaging system 100 includes a transmit beamformer 101 and a transmitter 102 that drives transducer elements 104 within a transducer array, herein referred to as an ultrasound probe 106, to emit pulsed ultrasonic signals (referred to herein as transmit pulses) into an imaging subject body (e.g., as further described with respect to FIG. 4). According to an embodiment, the ultrasound probe 106 may be a 1.5-dimensional (1.5D) probe, a 3D probe, or any ultrasound probe capable of live 3D imaging, such as a matrix array probe. The transducer elements 104 may be comprised of a piezoelectric material. When a voltage is applied to the piezoelectric material, the piezoelectric material physically expands and contracts, emitting an ultrasonic spherical wave. In this way, the transducer elements 104 may convert electronic transmit signals into acoustic transmit beams.

After the transducer elements 104 of the ultrasound probe 106 emit pulsed ultrasonic signals into a body (of a patient), the pulsed ultrasonic signals are back-scattered from structures within an interior of the body, like blood cells or muscular tissue, to produce echoes that return to the transducer elements 104. The echoes are converted into electrical signals, or ultrasound data, by the transducer elements 104, and the electrical signals are received by a receiver 108. The electrical signals representing the received echoes are passed through a receive beamformer 110 that performs beamforming and outputs ultrasound data, which may be in the form of a radiofrequency (RF) signal. Additionally, the transducer elements 104 may produce one or more ultrasonic pulses to form one or more transmit beams in accordance with the received echoes.

According to some embodiments, the ultrasound probe 106 may contain electronic circuitry to do all or part of the transmit beamforming and/or the receive beamforming. For example, all or part of the transmit beamformer 101, the transmitter 102, the receiver 108, and the receive beamformer 110 may be positioned within the ultrasound probe 106. The terms “scan” or “scanning” may also be used in this disclosure to refer to acquiring data through the process of transmitting and receiving ultrasonic signals. The term “data” may be used in this disclosure to refer to one or more datasets acquired with an ultrasound imaging system.

A user interface 115 may be used to control operation of the ultrasound imaging system 100, including to control the input of patient data (e.g., patient medical history), to change a scanning or display parameter, to initiate a probe repolarization sequence, and the like. The user interface 115 may include one or more of a rotary element, a mouse, a keyboard, a trackball, hard keys linked to specific actions, soft keys that may be configured to control different functions, and a graphical user interface displayed on a display device 118. In some embodiments, the display device 118 may include a touch-sensitive display, and thus, the display device 118 may be included in the user interface 115.

The ultrasound imaging system 100 also includes a processor 116 to control the transmit beamformer 101, the transmitter 102, the receiver 108, and the receive beamformer 110. The processor 116 is in electronic communication (e.g., communicatively connected) with the ultrasound probe 106. As used herein, the term “electronic communication” may be defined to include both wired and wireless communications. The processor 116 may control the ultrasound probe 106 to acquire data according to instructions stored on a memory of the processor and/or a memory 120. As one example, the processor 116 controls which of the transducer elements 104 are active and the shape of a beam emitted from the ultrasound probe 106. The processor 116 is also in electronic communication with the display device 118, and the processor 116 may process the data (e.g., ultrasound data) into images for display on the display device 118. The processor 116 may include a central processing unit (CPU), according to an embodiment. According to other embodiments, the processor 116 may include other electronic components capable of carrying out processing functions, such as a digital signal processor, a field-programmable gate array (FPGA), or a graphic board. According to other embodiments, the processor 116 may include multiple electronic components capable of carrying out processing functions. For example, the processor 116 may include two or more electronic components selected from a list of electronic components including: a central processor, a digital signal processor, a field-programmable gate array, and a graphic board. According to another embodiment, the processor 116 may also include a complex demodulator (not shown) that demodulates RF data and generates raw data. In another embodiment, the demodulation can be carried out earlier in the processing chain.

The processor 116 is adapted to perform one or more processing operations according to a plurality of selectable ultrasound modalities on the data. In one example, the data may be processed in real-time during a scanning session as the echo signals are received by receiver 108 and transmitted to processor 116. For the purposes of this disclosure, the term “real-time” is defined to include a procedure that is performed without any intentional delay (e.g., substantially at the time of occurrence). For example, an embodiment may acquire images at a real-time rate of 7-20 frames/sec. The ultrasound imaging system 100 may acquire 3D ultrasound image data of one or more planes at a significantly faster rate. However, it should be understood that the real-time frame-rate may be dependent on a length (e.g., duration) of time that it takes to acquire and/or process each frame of data for display. Accordingly, when acquiring a relatively large amount of data, the real-time frame-rate may be slower. Thus, some embodiments may have real-time frame-rates that are considerably faster than 20 frames/see while other embodiments may have real-time frame-rates slower than 7 frames/sec.

In some embodiments, the data may be stored temporarily in a buffer (not shown) during a scanning session and processed in less than real-time in a live or off-line (e.g., freeze) operation. Some embodiments of the disclosure may include multiple processors (not shown) to handle the processing tasks that are handled by the processor 116 according to the exemplary embodiment described hereinabove. For example, a first processor may be utilized to demodulate and decimate the RF signal while a second processor may be used to further process the data by augmenting the data as described further herein, prior to displaying an image. It should be appreciated that other embodiments may use a different arrangement of processors.

The ultrasound imaging system 100 may continuously acquire data at a frame-rate of, for example, 10 Hz to 30 Hz (e.g., 10 to 30 frames per second). Images generated from the data may be refreshed at a similar frame-rate on the display device 118. Other embodiments may acquire and display data at different rates. For example, some embodiments may acquire data at a frame-rate of less than 10 Hz or greater than 30 Hz depending on the size of the frame and the intended application. The memory 120 may store processed frames of acquired data. In an exemplary embodiment, the memory 120 is of sufficient capacity to store at least several seconds' worth of frames of ultrasound data. The frames of data are stored in a manner to facilitate retrieval thereof according to its order or time of acquisition. The memory 120 may comprise any known data storage medium.

In various embodiments of the present disclosure, data may be processed in different mode-related modules by the processor 116 to dynamic 3D ultrasound images. When multiple images are obtained, the processor 116 may also be configured to stabilize or register the images. For example, one or more modules may generate B-mode, color Doppler, M-mode, color M-mode, color flow imaging, spectral Doppler, elastography, tissue velocity imaging (TVI), strain, strain rate, and the like, and combinations thereof. As one example, the one or more modules may process color Doppler data, which may include traditional color flow Doppler, power Doppler, high-definition (HD) flow Doppler, and the like. The image lines and/or frames are stored in memory and may include timing information indicating a time at which the image lines and/or frames were stored in memory. The modules may include, for example, a scan conversion module to perform scan conversion operations to convert the acquired images from beam space coordinates to display space coordinates. A video processor module may be provided that reads the acquired images from a memory and displays an image in real-time while a procedure (e.g., ultrasound imaging) is being performed on a patient. The video processor module may include a separate image memory, and the ultrasound images may be written to the image memory in order to be read and displayed by the display device 118.

Further, the components of the ultrasound imaging system 100 may be coupled to one another to form a single structure, may be separate but located within a common room, or may be remotely located with respect to one another. For example, one or more of the modules described herein may operate in a data server that has a distinct and remote location with respect to other components of the ultrasound imaging system 100, such as the ultrasound probe 106 and the user interface 115. Optionally, the ultrasound imaging system 100 may be a unitary system that is capable of being moved (e.g., portably) from room to room. For example, the ultrasound imaging system 100 may include wheels or may be transported on a cart, or may comprise a handheld device.

For example, in various embodiments of the present disclosure, one or more components of the ultrasound imaging system 100 may be included in a portable, handheld ultrasound imaging device. For example, the display device 118 and the user interface 115 may be integrated into an exterior surface of the handheld ultrasound imaging device, which may further contain the processor 116 and the memory 120 therein. The ultrasound probe 106 may comprise a handheld probe in electronic communication with the handheld ultrasound imaging device to collect raw ultrasound data. The transmit beamformer 101, the transmitter 102, the receiver 108, and the receive beamformer 110 may be included in the same or different portions of the ultrasound imaging system 100. For example, the transmit beamformer 101, the transmitter 102, the receiver 108, and the receive beamformer 110 may be included in the handheld ultrasound imaging device, the probe, and combinations thereof.

Referring to FIG. 2, an example medical image processing system 200 is shown. In some embodiments, the medical image processing system 200 is incorporated into a medical imaging system, such as an ultrasound imaging system (e.g., the ultrasound imaging system 100 of FIG. 1), an MRI system, a CT system, a single-photon emission computed tomography (SPECT) system, etc. In some embodiments, at least a portion of the medical image processing system 200 is disposed at a device (e.g., an edge device or server) communicably coupled to the medical imaging system via wired and/or wireless connections. In some embodiments, the medical image processing system 200 is disposed at a separate device (e.g., a workstation) that can receive images from the medical imaging system or from a storage device that stores the images generated by the medical imaging system. The medical image processing system 200 may comprise an image processor 231, a user input device 232, and a display device 233. For example, the image processor 231 may be operatively/communicatively coupled to the user input device 232 and the display device 233.

The image processor 231 includes a processor 204 configured to execute machine-readable instructions stored in non-transitory memory 206. The processor 204 may be single core or multi-core, and the programs executed by the processor 204 may be configured for parallel or distributed processing. In some embodiments, the processor 204 may optionally include individual components that are distributed throughout two or more devices, which may be remotely located and/or configured for coordinated processing. In some embodiments, one or more aspects of the processor 204 may be virtualized and executed by remotely-accessible networked computing devices configured in a cloud computing configuration. In some embodiments, the processor 204 may include other electronic components capable of carrying out processing functions, such as a digital signal processor, a field-programmable gate array (FPGA), or a graphics board. In some embodiments, the processor 204 may include multiple electronic components capable of carrying out processing functions. For example, the processor 204 may include two or more electronic components selected from a plurality of possible electronic components, including a central processor, a digital signal processor, a field-programmable gate array, and a graphics board. In still further embodiments, the processor 204 may be configured as a graphical processing unit (GPU), including parallel computing architecture and parallel processing capabilities.

In the embodiment shown in FIG. 2, the non-transitory memory 206 stores a 3D generation module 212 and medical image data 214. The 3D generation module 212 includes one or more algorithms to process input medical images from the medical image data 214. Specifically, the 3D generation module 212 may generate a dynamic 3D ultrasound image from dynamic 3D ultrasound image data, as described with respect to FIGS. 5 and 6. For example, the 3D generation module 212 may include one or more image recognition algorithms, shape or edge detection algorithms, gradient algorithms, and the like to process the 3D ultrasound image data. Additionally or alternatively, the 3D generation module 212 may store instructions for implementing a neural network, such as a convolutional neural network, for detecting pleural surfaces/pleura captured in the medical image data 214 in real-time. For example, the 3D generation module 212 may include trained and/or untrained neural networks and may further include training routines, or parameters (e.g., weights and biases), associated with one or more neural network models stored therein. In some embodiments, the 3D generation module 212 may evaluate the medical image data 214 as it is acquired in real-time. Additionally or alternatively, the 3D generation module 212 may evaluate the medical image data 214 offline, not in real-time.

As an example, when the medical image data 214 includes lung ultrasound data, the identified anatomical feature may include lung pleura, which may be identified by the 3D generation module 212 based on pleural sliding via edge detection techniques and/or gradient changes. Additionally, the 3D generation module 212 may identify a plurality of rib spaces via edge detection techniques and/or gradient changes. As will be elaborated with respect to FIGS. 5 and 6, detection of rib spaces may assist in generating a plurality of rib space segments, and detection of pleural positioning may assist in indicating a region of an ultrasound image which is noise and may be removed to expose a pleural surface.

Optionally, the image processor 231 may be communicatively coupled to a training module 210, which includes instructions for training one or more of the machine learning models stored in the 3D generation module 212. The training module 210 may include instructions that, when executed by a processor, cause the processor to build a model (e.g., a mathematical model) based on sample data to make predictions or decisions regarding the detection and classification of anatomical irregularities without the explicit programming of a conventional algorithm that does not utilize machine learning. In one example, the training module 210 includes instructions for receiving training data sets from the medical image data 214. The training data sets comprise sets of medical images, associated ground truth labels/images, and associated model outputs for use in training one or more of the machine learning models stored in the 3D generation module 212. The training module 210 may receive medical images, associated ground truth labels/images, and associated model outputs for use in training the one or more machine learning models from sources other than the medical image data 214, such as other image processing systems, the cloud, etc. In some embodiments, one or more aspects of the training module 210 may include remotely-accessible networked storage devices configured in a cloud computing configuration. Further, in some embodiments, the training module 210 is included in the non-transitory memory 206. Additionally or alternatively, in some embodiments, the training module 210 may be used to generate the 3D generation module 212 offline and remote from the image processing system 200. In such embodiments, the training module 210 may not be included in the image processing system 200 but may generate data stored in the image processing system 200. For example, the 3D generation module 212 may be pre-trained with the training module 210 at a place of manufacture.

The non-transitory memory 206 further stores the medical image data 214. The medical image data 214 includes, for example, functional and/or anatomical images captured by an imaging modality, such as an ultrasound imaging system, an MRI system, a CT system, a PET system, etc. As one example, the medical image data 214 may include ultrasound images, such as lung ultrasound images. Further, the medical image data 214 may include one or more of 2D images, 3D images, static single frame images, and multi-frame cine-loops (e.g., movies).

In some embodiments, the non-transitory memory 206 may include components disposed at two or more devices, which may be remotely located and/or configured for coordinated processing. In some embodiments, one or more aspects of the non-transitory memory 206 may include remotely-accessible networked storage devices in a cloud computing configuration. As one example, the non-transitory memory 206 may be part of a picture archiving and communication system (PACS) that is configured to store patient medical historics, imaging data, test results, diagnosis information, management information, and/or scheduling information, for example.

The image processing system 200 may further include the user input device 232. The user input device 232 may comprise one or more of a touchscreen, a keyboard, a mouse, a trackpad, a motion sensing camera, or other device configured to enable a user to interact with and manipulate data stored within the image processor 231.

The display device 233 may include one or more display devices utilizing any type of display technology. In some embodiments, the display device 233 may comprise a computer monitor and may display unprocessed images, processed images, parametric maps, and/or exam reports. The display device 233 may be combined with the processor 204, the non-transitory memory 206, and/or the user input device 232 in a shared enclosure or may be a peripheral display device. The display device 233 may include a monitor, a touchscreen, a projector, or another type of display device, which may enable a user to view medical images and/or interact with various data stored in the non-transitory memory 206. In some embodiments, the display device 233 may be included in a smartphone, a tablet, a smartwatch, or the like.

It may be understood that the medical image processing system 200 shown in FIG. 2 is one non-limiting embodiment of an image processing system, and other imaging processing systems may include more, fewer, or different components without departing from the scope of this disclosure. Further, in some embodiments, at least portions of the medical image processing system 200 may be included in the ultrasound imaging system 100 of FIG. 1, or vice versa (e.g., at least portions of the ultrasound imaging system 100 may be included in the medical image processing system 200).

FIG. 3 illustrates an embodiment of the ultrasound probe 106 of the ultrasound imaging system 100 of FIG. 1. The ultrasound probe 106 includes a housing 300 that may house transducer elements 104 (not shown in FIG. 3). The housing 300 may be in contact with an imaging subject (e.g., a patient) along a face surface 302 of the housing 300. The face surface 302 of the housing 300 may have a substantially rectangular shape that is elongated along a first edge 304 relative to a second, perpendicular edge 306.

The ultrasound probe 106 may be moved (e.g., in a sweeping motion) along an outside surface of the imaging subject to acquire ultrasound image data of interior structured of the imaging subject. For example, the imaging subject may be a patient, and the ultrasound probe 106 may be moved across a thoracic cavity of the patient to acquire ultrasound image data of lungs and ribs of the patient. A user, such as an operator of the ultrasound imaging system 100, may physically sweep the ultrasound probe 106 across multiple rib spaces of the thoracic cavity of a patient while acquiring 3D data sets. In some embodiments, a dynamic 3D ultrasound image may be gradually generated in real-time as the ultrasound probe 106 is swept by the user. Further detail regarding acquisition of lung and rib ultrasound image data is described with respect to FIG. 4. Generation of the dynamic 3D ultrasound image is described with respect to FIGS. 5-10. Ultrasonic signals may be pulsed from the transducer elements 104 into a body of the subject, and the pulsed ultrasonic signals are back-scattered from structures within an interior of the body to produce echoes that return to the transducer elements 104 housed in the housing 300. As described with respect to FIG. 1, the ultrasound probe 106 may be a 1.5D probe, a 3D probe, or any ultrasound probe capable of live 3D imaging, such as a matrix array probe.

As used herein, the terms “system” and “module” may include a hardware and/or software system that operates to perform one or more functions. For example, a module or system may include or may be included in a computer processor, controller, or other logic-based device that performs operations based on instructions stored on a tangible and non-transitory computer readable storage medium, such as a computer memory. Alternatively, a module or system may include a hard-wired device that performs operations based on hard-wired logic of the device. Various modules or systems shown in the attached figures may represent the hardware that operates based on software or hardwired instructions, the software that directs hardware to perform the operations, or a combination thereof.

“Systems” or “modules” may include or represent hardware and associated instructions (e.g., software stored on a tangible and non-transitory computer readable storage medium, such as a computer hard drive, ROM, RAM, or the like) that perform one or more operations described herein. The hardware may include electronic circuits that include and/or are connected to one or more logic-based devices, such as microprocessors, processors, controllers, or the like. These devices may be off-the-shelf devices that are appropriately programmed or instructed to perform operations described herein from the instructions described above. Additionally or alternatively, one or more of these devices may be hard-wired with logic circuits to perform these operations.

FIG. 4 illustrates a thoracic cavity 400 of a patient 404, according to an embodiment. Ultrasound image data acquired by the ultrasound probe 106 (e.g., of FIGS. 1 and 3) and displayed as a dynamic 3D ultrasound image may represent portions of the thoracic cavity 400, including lungs 408, a plurality of ribs, and a sternum 410 of the patient 404. The plurality of ribs may include, from a bottom 442 to a top 440 of the thoracic cavity 400: a first rib 421, a second rib 422, a third rib 423, a fourth rib 424, a fifth rib 425, a sixth rib 426, a seventh rib 427, an eighth rib 428, a ninth rib 429, and a tenth rib 430. FIG. 4 also shows a plurality of intercostal spaces, also referred to herein as rib spaces, located between the ribs. For example, a first intercostal space 431, a second intercostal space 432, a third intercostal space 433, a fourth intercostal space 434, and a fifth intercostal space 435 are represented in FIG. 4. The first intercostal space 431 is located between the first rib 421 and the second rib 422, the second intercostal space 432 is located between the second rib 422 and the third rib 423, the third intercostal space 433 is located between the third rib 423 and the fourth rib 424, the fourth intercostal space 434 is located between the fourth rib 424 and the fifth rib 425, and the fifth intercostal space 435 is located between the fifth rib 425 and the sixth rib 426. The thoracic cavity 400 includes additional intercostal spaces which are not specifically identified in FIG. 4.

The ultrasound probe 106 of FIGS. 1-3 may generally be held by a user in an orientation where the edge 306 of the housing 300 is oriented parallel (e.g., more parallel than perpendicular) to the plurality of ribs. The ultrasound probe 106 of FIGS. 1 and 3 may be moved transversely to directions in which the plurality of ribs are elongated. For example, the ultrasound probe 106 may be moved along a direction substantially parallel to the sagittal plane 402. The ultrasound probe 106 may be moved along an exterior of the patient 404 in directions that are more parallel than perpendicular with respect to the sagittal plane 402. This orientation of the ultrasound probe 106 may be referred to as a sagittal position or orientation. Alternatively, the ultrasound probe 106 may be held by a user in an orientation that is perpendicular to the sagittal orientation, such that the edge 306 is perpendicular to the plurality of ribs while the ultrasound probe 106 is moved along a direction substantially parallel to the sagittal plane 402. This orientation may be referred to as a transverse position or orientation of the ultrasound probe 106.

FIG. 5 shows a flow chart for a method 500 for generating a dynamic 3D ultrasound image. In particular, the method 500 provides a workflow for generating a dynamic 3D ultrasound image of ribs, rib spaces, and other anatomies of a thoracic cavity of an imaging subject. For example, the method 500 may be a high-level method used to generate a dynamic image of elements of the thoracic cavity 400 and rib spaces (e.g., the first intercostal space 431, the second intercostal space 432, and so on) of the patient 404 described with respect to FIG. 4. The method 500 will be described for dynamic 3D ultrasound images acquired using an ultrasound imaging system, such as the ultrasound imaging system 100 of FIG. 1, although other ultrasound imaging systems may be used. Further, the method 500 may be adapted to other imaging modalities. The method 500 may be implemented by one or more of the above described systems, including the ultrasound imaging system 100 of FIG. 1, the medical image processing system 200 of FIG. 2, and/or the ultrasound probe 106 of FIG. 3, as further described herein. Briefly, instructions for executing the method 500 may be stored as computer-readable instructions in non-transitory memory, such as the memory 120 of FIG. 1 and/or the non-transitory memory 206 of FIG. 2, and executed by a processor, such as the processor 116 of FIG. 1 and/or the processor 204 of FIG. 2. Further, in some embodiments, the method 500 is performed in real-time (e.g., as 3D ultrasound image data) is captured (e.g., using an ultrasound probe, such as the ultrasound probe 106 of FIG. 3). For example, the method 500 may execute operations described herein (e.g., generating a plurality of 3D rib space segments, and so on) as the dynamic 3D ultrasound image data is captured using the ultrasound probe. In other embodiments, at least portions of the method 500 are performed offline, after conclusion of an imaging scan performed to capture the 3D ultrasound image data. For example, the processor may evaluate 3D ultrasound image data that are stored in memory even while the ultrasound system is not actively being operated to acquire 3D ultrasound image data.

At 502, the method 500 includes acquiring dynamic 3D ultrasound image data, herein also “image data”. As described above, the 3D ultrasound image data may be acquired in real-time or near real-time. In another embodiment, dynamic 3D ultrasound image data may be captured during an imaging scan and stored in a memory and/or data base for later use, and acquiring dynamic 3D ultrasound data may include requesting and receiving the dynamic 3D ultrasound data from the database. The 3D ultrasound image data may be acquired using a 3D ultrasound probe, such as the ultrasound probe 106 described with respect to FIGS. 1 and 3. As described above, an imaging scan used to capture 3D ultrasound image data may include a longitudinal scan, an oblique scan, and/or a panoramic sweep. In the longitudinal scan, the ultrasound probe is positioned perpendicular to the ribs. In the oblique scan, the ultrasound probe is positioned along intercostal spaces between the ribs. In the panoramic sweep, the ultrasound probe may be swept (e.g., by a user) from a cranial side to a caudal side of the thoracic cavity, along the sagittal plane, a described with respect to FIG. 4. A user, such as an operator of the ultrasound imaging system 100, may physically sweep the ultrasound probe 106 across multiple rib spaces of the thoracic cavity of a patient while acquiring 3D data sets. In some embodiments, a dynamic 3D ultrasound image may be gradually generated in real-time as the ultrasound probe 106 is swept by the user, as further described herein. A preferred sweeping motion for capturing image data of rib spaces, as described herein, is a sagittal motion that moves downwards from a top rib (e.g., the tenth rib 430) to a bottom rib (e.g., the first rib 421), or upwards from the bottom rib to the top rib. In some embodiments, the ultrasound probe 106 may be swept over a portion of the thoracic cavity 400, for example, across multiple rib spaces in a sagittal motion from the bottom 442 of the thoracic cavity 400 to a top 440 of the thoracic cavity 400, or vice versa. As further described herein, multiple views from the imaging scan may be stitched together to provide anatomical and spatial relationships of rib space segments captured in the dynamic 3D ultrasound image data.

The 3D ultrasound probe may continuously capture 3D ultrasound image data, and features of the 3D ultrasound image data may change during the imaging scan. For example, structures of a lung pleura may change with the imaging subject's breathing cycles, movement, and so on. The acquired 3D ultrasound image data is dynamic image data, such as a video showing movement of one or more portions of the rib spaces and/or rib shadows. This movement may result in one or more features of interest appearing at times and disappearing from the 3D ultrasound image data at other times. For example, B-lines or other features in the image data that may indicate pneumonia infection, air bronchograms, or other damage may be visible in some, but not all, of the image frames.

As described above, the dynamic 3D ultrasound image data may be captured by sweeping the ultrasound probe over the thoracic cavity of an imaging subject. During the imaging scan, the ultrasound probe may be moved over different areas of the thoracic cavity such that different amounts of image data are captured for different regions of the thoracic cavity. For example, the ultrasound probe may be swept over the first rib and first space, the second rib and the second space, and the third rib and the third space. The ultrasound probe may then be positioned over the third rib and/or the third rib space. The ultrasound probe may be swept further down the thoracic cavity (e.g., parallel to the sagittal plane 402), and/or may be moved back towards a top of the thoracic cavity to capture additional image data of the first rib, the first rib space, and so on. In this example, acquired dynamic 3D ultrasound image data may be a continuous video, for example, that starts at a top of the thoracic cavity, progresses downwards, lingers in the middle of the thoracic cavity, moves back upwards, continues downwards, and so on to capture image data. Therefore, more image data may be captured of some regions/structures compared to others.

A live view of the 3D ultrasound image data (e.g., volumetric structure of internal structures of the imaging subject) may show structures within the FOV of the ultrasound probe at a current position of the ultrasound probe. The FOV of the ultrasound probe used to capture image data of a thoracic cavity of a patient may be sized to capture one to two rib spaces at a time. It may be desirable to view more structures than are shown in the FOV (e.g., more of the present rib shown, rib spaces on either side, ribs on either side, etc.) which may assist in diagnostic analysis. Presently, visualization of additional regions demands movement of the ultrasound probe to capture the additional regions in the ultrasound probe FOV. However, it may be desirable to simultaneously view multiple regions, including regions which may not be able to be viewed at the same time within size constraints of the ultrasound probe FOV. An example of a 3D ultrasound image captured within a FOV of an ultrasound probe is shown in FIG. 7.

At 504, the method 500 includes generating a plurality of 3D rib space segments from the dynamic 3D ultrasound image data. Each rib space segment of the plurality of rib space segments may be a region of interest including a rib space (e.g., interstitial space between two ribs), and at least part of each of the ribs on either side of the rib space. As described above, the 3D ultrasound image data captured during an imaging scan may be obtained and/or stored in the order in which it is collected, which may or may not be the same as an anatomical order of the imaging subject. In some embodiments, each 3D rib space segment of the plurality of 3D rib space segments may be generated in real-time as the ultrasound probe 106 is swept by the user over the corresponding rib space. Additionally, the FOV of the ultrasound probe may capture more than one rib space and bordering ribs thereof. Individual rib space segments in the 3D ultrasound image data may be identified using one or more methods. For example, the processor may be configured to automatically examine characteristics of the pixels, and/or other subsets of the image data, to identify the rib space segments, such as the color, intensity, brightness, or the like, of pixels in the image data. The processor may further divide the dynamic 3D ultrasound image data into multiple 3D rib space segments based on the characteristics of the different structures (e.g., a curvature of the rib(s), a width of the rib space(s)). In another example, a center of a FOV of the ultrasound probe may be tracked during the imaging scan, and a relative position of the ultrasound probe on the surface of the thoracic cavity with respect to structures (e.g., ribs and rip spaces) may be used to automatically identify regions of interest. Further detail regarding generating of rib space segments is described with respect to the method 600 of FIG. 6. Examples of annotated dynamic 3D ultrasound images are shown in FIG. 7.

At 506, the method 500 includes generating a dynamic panoramic 3D ultrasound image depicting the plurality of 3D rib space segments in an anatomical order. Characteristics of the dynamic 3D ultrasound image data used to generate the plurality of 3D rib space segments may further be used to identify specific structures shown in each 3D rib space segment. For example, a curvature of the rib(s), a width of the rib space(s), a relative position of the ultrasound probe, and/or structures captured in the FOV of the ultrasound probe may be automatically examined to identify which rib and/or rib space (e.g., the first rib 421, the first intercostal space 431, the second rib 422, and so on) of the thoracic cavity is shown in each 3D rib space segment. The plurality of 3D rib space segments may be mapped to a reference thoracic cavity, or other anatomical reference, to order each of the plurality of 3D rib space segments according to the anatomical order. As briefly described above, dynamic 3D ultrasound image data may be captured of the ribs and rib spaces in an order that is different from the anatomical order. Thus, identifying specific rib and rib space structures in the plurality of 3D rib space segments and ordering the plurality of 3D rib space segments accordingly presents the dynamic 3D ultrasound image data in a true-to-anatomy configuration. This may enable visualization of irregularities and/or pathologies that may extend between/through multiple rib spaces, and/or that may be variably visible, depending on a time point and a perspective view of the dynamic 3D ultrasound image data. Once the plurality of 3D rib space segments are positioned in the anatomical order, the 3D rib space segmented may be joined (e.g., stitched) to form a single, dynamic 3D ultrasound image, where the plurality of 3D rib space segments are continuous (e.g., without gaps or breaks therebetween, other than rib spaces of the anatomy).

At 508, the method 500 includes temporally synchronizing the plurality of dynamic 3D rib space segments. As described above, the dynamic 3D ultrasound image data of each 3D rib space segment may have a different duration (e.g., a different period) and/or may include a different number of image frames. Generation of the dynamic 3D ultrasound image includes temporally synchronizing the plurality of dynamic 3D rib space segments such that the dynamic 3D ultrasound image has a single duration to which the dynamic 3D rib space segments are synchronized. Temporal scaling may be applied to one or more of the dynamic 3D rib space segments to expand or contract one or more of the dynamic 3D rib space segments. For example, a first dynamic 3D rib space segment may be temporally expanded such that the dynamic 3D image data thereof takes a longer amount of time to play, and a second dynamic 3D rib space segment may be temporally contracted such that the dynamic 3D image data thereof takes a shorter amount of time to play. The durations of the first dynamic 3D rib space segment and the second dynamic 3D rib space segment may be equal following temporal scaling. Temporally expanding a dynamic 3D rib space segment results in the associated dynamic 3D ultrasound image data playing at a slower frame rate than an acquisition frame rate, and temporally contracting a dynamic 3D rib space segment results in the associated dynamic 3D ultrasound image data playing at a faster frame rate than the acquisition frame rate.

The processor may utilize additional techniques either in addition to or in place of temporally contracting or expanding the dynamic 3D ultrasound image data of one or more dynamic 3D rib space segments to perform temporal scaling thereof. For example, if more dynamic 3D ultrasound image data is captured for the first dynamic 3D rib space segment compared to the second dynamic 3D rib space segment, dynamic 3D ultrasound image data of the second dynamic 3D rib space segment may have fewer frames than dynamic 3D ultrasound image data of the first dynamic 3D rib space segment. Temporal scaling may include playing the dynamic 3D ultrasound image data of the dynamic 3D rib space segment having fewer frames more than one time, while dynamic 3D ultrasound image data of the dynamic 3D rib space segment having more frames may be played a single time. For example, if the first dynamic 3D rib space segment has ten frames and the second dynamic 3D rib space segment has thirty frames, the processor may play dynamic 3D ultrasound image data of the first dynamic 3D rib space segment three times, and play dynamic 3D ultrasound image data of the second dynamic 3D rib space segment a single time to provide appropriate temporal scaling of each dynamic 3D rib space segment.

At 510, the method 500 includes outputting the dynamic panoramic 3D ultrasound image for display. In some examples, the display is included in the ultrasound imaging system, such as display device 118. Example displays of the dynamic panoramic 3D ultrasound image are shown in FIGS. 8-10. The dynamic panoramic 3D ultrasound image may be saved with and/or without annotations (e.g., pleural line indicators) in some examples. Further, raw, unprocessed ultrasound data may be saved, at least in some examples. The memory may be local to the ultrasound imaging system or may be a remote memory. For example, the unannotated and annotated images may be saved and/or archived (e.g., as a structured report in a PACS system) so that they may be retrieved and used to generate an official, physician-signed report that may be included in the patient's medical record. Thus, generating the dynamic panoramic 3D ultrasound image may simplify image analysis by showing detailed topography of the pleura in one image (e.g., the dynamic panoramic 3D ultrasound image), rather than in multiple dynamic 3D ultrasound images.

FIG. 6 shows a flow chart of a method 600 for generating a dynamic 3D ultrasound image from 3D ultrasound image data. The method 600 provides further detail and may be an embodiment of the method 500 of FIG. 5. The method 600 is described at least partially with respect to FIG. 5, as well as with reference to FIGS. 7-10, as will be further described following the description of the method 600. The method 600 will be described for dynamic 3D ultrasound images acquired using an ultrasound imaging system, such as the ultrasound imaging system 100 of FIG. 1, although other ultrasound imaging systems may be used. Further, the method 600 may be adapted to other imaging modalities. The method 600 may be implemented by one or more of the above described systems, including the ultrasound imaging system 100 of FIG. 1, the medical image processing system 200 of FIG. 2, and/or the ultrasound probe 106 of FIG. 3, as further described herein. Briefly, instructions for executing the method 600 may be stored as computer-readable instructions in non-transitory memory, such as the memory 120 of FIG. 1 and/or the non-transitory memory 206 of FIG. 2, and executed by a processor, such as the processor 116 of FIG. 1 and/or the processor 204 of FIG. 2. Further, in some embodiments, the method 600 is performed in real-time (e.g., as 3D ultrasound image data) is captured (e.g., using an ultrasound probe, such as the ultrasound probe 106 of FIG. 3). For example, the method 600 may execute operations described herein (e.g., generating a plurality of 3D rib space segments, and so on) as the dynamic 3D ultrasound image data is captured using the ultrasound probe. In other embodiments, at least portions of the method 600 are performed offline, after conclusion of an imaging scan performed to capture the 3D ultrasound image data. For example, the processor may evaluate 3D ultrasound image data that are stored in memory even while the ultrasound system is not actively being operated to acquire 3D ultrasound image data.

At 602, the method 600 includes acquiring dynamic 3D ultrasound image data, herein also “image data”. Dynamic 3D ultrasound image data may be acquired as described with respect to operation 502 of the method 500. Briefly, the 3D ultrasound image data may be acquired in real-time, near real-time, or may be captured during an imaging scan and stored in a memory and/or data base for later use, and acquiring dynamic 3D ultrasound data may include requesting and receiving the dynamic 3D ultrasound data from the database. The 3D ultrasound image data may be acquired using a 3D ultrasound probe, such as the ultrasound probe 106 described with respect to FIGS. 1 and 3.

At 604, the method 600 includes identifying a plurality of rib spaces of the dynamic 3D ultrasound image data. As described with respect to operation 504 of FIG. 5, the method 500 includes generating a plurality of 3D rib space segments from the dynamic 3D ultrasound image data. Operation 604 includes identifying the plurality of rib spaces, which may include separating the dynamic 3D ultrasound image data into the plurality of rib space segments (e.g., where each rib space segment of the plurality of rib space segments includes a rib space of the plurality of rib spaces). As described with respect to the method 500, the FOV of the ultrasound probe may simultaneously capture more than one rib space and bordering ribs thereof. Individual rib space segments in the 3D ultrasound image data may be identified using one or more methods. For example, the processor may be configured to automatically examine characteristics of the pixels, and/or other subsets of the image data, to identify the rib space segments, such as the color, intensity, brightness, or the like, of pixels in the image data. The processor may further divide the dynamic 3D ultrasound image data into multiple 3D rib space segments based on the characteristics of the different structures (e.g., a curvature of the rib(s), a width of the rib space(s)). In another example, a center of a FOV of the ultrasound probe may be tracked during the imaging scan, and a relative position of the ultrasound probe on the surface of the thoracic cavity with respect to structures (e.g., ribs and rip spaces) may be used to automatically identify regions of interest.

The 3D ultrasound image data includes a plurality of frames of 3D ultrasound image data, each acquired at a different time. As long as the ultrasound probe is being translated during acquisition of the ultrasound image data, each of the image frames may be acquired from a different spatial position with respect to the imaging subject's anatomy. The processor may be further configured to identify time stamps or other identifiers in the 3D ultrasound image data associated with a transition from an intercostal space to rib shadow. For example, intensities associated with a rib shadow may be relatively low, and intensities associated with intercostal spaces are relatively high. As the ultrasound probe is being translated during acquisition of 3D ultrasound image data, anatomy being acquired in each frame is different. The processor may be configured to identify the frame in the 3D ultrasound image data where the 3D ultrasound image data transitions from a relatively low intensity (e.g., associated with a rib shadow) to a relatively high intensity (e.g., associated with a particular intercostal space). Further, the processor may be configured to identify the frame in the 3D ultrasound image data where the 3D ultrasound image data transitions from a particular intercostal space with a relatively high intensity to an adjacent (e.g., next) rib shadow with a relatively low intensity.

At 606, the method 600 includes identifying and exposing a pleural surface of each of the plurality of rib spaces. In an aerated lung, the pleura, which form the outer boundary of the lung that lies against the chest wall, may provide the anatomical lung structure substantially detectable by ultrasound. The pleura may appear as a hyperechoic horizontal segment of brighter (e.g., whiter) pixels in a 3D ultrasound image, referred to as a pleural line, which moves synchronously with a respiratory cycle in a phenomenon known as pleural sliding. A 3D pleural surface includes a volumetric topography of the lung pleura. Pleura topography may be visualized in a 3D pleural surface, which may help in identifying and/or diagnosing conditions of the lung that may be visualized as irregularities in pleural surface topography. Compared to what may be captured in a 2D ultrasound image, the 3D pleural surface may show pleural topography in a broader region of the lung. Additionally, in dynamic 3D ultrasound images, changes to the 3D pleural surface over time may be shown, where the changes reflect changes in patient anatomy visualized using the ultrasound probe (e.g., due to patient breathing, movement, etc.).

Detecting a bottom edge of the pleura (e.g., where the pleural surface ends and the interior of the lung begins) may include identifying lower and upper borders of the pleura based on a brightness difference among pixels, such as by using edge detection techniques or gradient changes. For example, the processor may apply an edge detection algorithm, which may be included in the 3D generation module 212 of FIG. 2. The edge detection algorithm may comprise one or more mathematical models for identifying points (e.g., pixels) at which the image brightness changes sharply and/or has discontinuities to identify the lower and upper borders of the pleural line. As one example, the processor may apply the edge detection algorithm to the area having the highest amount of local change. As another example, additionally or alternatively, a gradient algorithm may identify a local maximum and/or minimum pixel brightness at the pleural position to identify the lower and upper borders of the pleural line in each image. In some embodiments, a deep learning model trained for real-time or static (e.g., not real-time) pleura detection is used to identify the bottom edge of the pleura.

The lower and upper borders of the pleura may include sub pleural consolidations. For example, the bottom edge (e.g., indicating boundary below which to remove ultrasound data, as further described herein) may be positioned a predetermined distance towards the lung from the pleural line. It may be understood that, conventionally, sub pleural consolidations extend a maximum distance ‘n’ from the pleural surface towards the lung. Thus, the bottom edge of the pleura may be positioned a distance ‘n’ from the lower border of the pleura identified as described above. In other embodiments, sub pleural consolidations may be identified using the same or a different edge detection algorithm described above. An example 3D ultrasound image including indicators of the pleural line is described with respect to FIG. 7.

Following identification of the bottom edge of the pleural line, operation 606 of the method 600 includes removing ultrasound data from below the bottom edge of the pleural line in each 3D ultrasound image of the 3D ultrasound image data. As described with respect to FIG. 7, ultrasound image data vertically below the bottom edge of the pleural line may be noise or other imaging artifacts that do not indicate the topography of the pleural line. As described above, sub pleural consolidations are included in the boundary defined by the bottom edge. Removing ultrasound data may include generating a new image dataset for each 3D ultrasound image, the new image dataset including ultrasound data above and including the pleural line, and excluding data below the bottom edge of the pleural line. The 3D pleural surface is thus exposed in the 3D ultrasound image formed by the new image dataset. Operation 604 for detecting and exposing a pleural surface of the lung region in the 3D ultrasound image data may be performed for all of the plurality of rib spaces in the 3D ultrasound image data. In this way, the 3D pleural surface is exposed for each 3D ultrasound image of each rib space.

At 608, the method 600 includes sequentially aligning the plurality of rib spaces according to an anatomical order using image characteristics of the pleural surface. Characteristics of the structures in each of the plurality of rib spaces (e.g., a curvature of the rib(s), a width of the rib space(s)) may be used to identify the rib and/or rib space shown in each of the plurality of rib spaces. As described above, the plurality rib spaces may be mapped to a reference thoracic cavity, or other anatomical reference, to order each of the plurality rib spaces according to the anatomical order.

At 610, the method 600 includes synchronizing a timing of display of each rib space of the plurality of rub spaces such that a start time and an end time of display of each rib space occurs at the same time. The 3D ultrasound image data is dynamic, such as a video showing movement of one or more portions of the rib spaces and/or ribs. The groups of 3D ultrasound image data for different regions of interest may include 3D ultrasound image data of different durations. Temporally synchronizing the 3D ultrasound data includes identifying a target duration to be used in a dynamic 3D ultrasound image. For example, the processor may determine an acquisition duration for each of the regions of interest in the 3D ultrasound image data, and use the acquisition durations to identify the target duration. For example, intensity information from the 3D ultrasound image data may be used to identify a start time and an end time of when structures of interest in the 3D ultrasound image data are present in the region of interest. As described with respect to the method 500, the 3D ultrasound image data for one or more rib spaces may be temporally expanded or contracted.

In another example, synchronizing the timing of display of each rib space comprises adjusting a playback speed of display of one or more rib spaces. Adjusting the playback speed may include adjusting an amount of time that each frame of the dynamic 3D ultrasound image data for a given rib space is displayed, thus adjusting an overall playback speed for the given rib space. The playback speed may be adjusted by playing the dynamic 3D ultrasound image data of each frame of a first rib space having fewer frames for a longer duration, and playing the dynamic 3D ultrasound image data of each frame of a second rib space segment having more frames for a shorter duration. For example, each frame of a first rib space having ten frames may be played for three times as long (e.g., three times the duration) as each frame of a second rib space having thirty frames to provide appropriate temporal scaling of each rib space.

In a further example, synchronizing the timing of display comprises identifying a respiratory cycle in each of the plurality of rib spaces, and synchronizing the respiratory cycle of the plurality of rib spaces. As described above, segments of the dynamic 3D ultrasound image data may show different regions, structures, and/or abnormalities at different times during the imaging scan (e.g., at different parts of the respiratory cycle). Thus, it is desirable to synchronize the timing of display of the plurality of rib spaces by synchronizing the respiratory cycle of the plurality of rib spaces such that structures, pathologies, and/or configurations of anatomy that occur simultaneously in the imaging subject anatomy are shown simultaneously in the single, dynamic 3D ultrasound image. Synchronizing the respiratory cycle of the plurality of rib spaces includes simultaneously showing dynamic 3D ultrasound image data captured during a first inhalation for a first duration, and simultaneously showing the dynamic 3D ultrasound image data captured during a first exhalation for a second duration, separate from the first duration. For example, the respiratory cycle may include a series of alternating inhalations and exhalations. A part of the respiratory cycle (e.g., inhalation or exhalation) may be identified in the dynamic 3D ultrasound data using image characteristics such as the color, intensity, brightness, or the like, of pixels in the image data. For example, pixels of the pleural surface may be brighter during inhalation compared to brightness of pixels of the pleural surface during exhalation due to an expansion of lung tissue. The dynamic 3D ultrasound image data for each rib space may be synchronized such that the start and the end of display of each rib space occurs simultaneously. This may include adjusting the playback speed of one or more rib spaces, and/or temporally expanding and/or contracting the 3D ultrasound image data for one or more rib spaces.

At 612, the method 600 includes displaying the 3D ultrasound image data as a dynamic 3D ultrasound image. In some examples, the dynamic 3D ultrasound image display is displayed on a display included in the ultrasound imaging system, such as display device 118. Example displays of the dynamic panoramic 3D ultrasound image are shown in FIGS. 8-10. The dynamic panoramic 3D ultrasound image may be saved with and/or without annotations (e.g., pleural line indicators) in some examples. Further, raw, unprocessed ultrasound data may be saved, at least in some examples. The memory may be local to the ultrasound imaging system or may be a remote memory. For example, the unannotated and annotated images may be saved and/or archived (e.g., as a structured report in a PACS system) so that they may be retrieved and used to generate an official, physician-signed report that may be included in the patient's medical record. Thus, generating the dynamic panoramic 3D ultrasound image may simplify image analysis by showing detailed topography of the pleura in one image (e.g., the dynamic panoramic 3D ultrasound image), rather than in multiple dynamic 3D ultrasound images.

FIG. 7 shows an example annotated 3D rib space segment 702 that may be used to generate a 3D pleural surface image 704. Each of the annotated 3D rib space segment 702 and the 3D pleural surface image 704 are described herein with reference to FIGS. 1-6, and includes lungs 408, ribs (e.g., the first rib 421), and rib spaces (e.g., the first intercostal space 431) of the patient 404 acquired with the ultrasound probe 106 held in a sagittal orientation (e.g., performing a panoramic sweep). The rib shadows indicate locations where passage of the pulsed ultrasonic signals was blocked by the ribs.

The annotated 3D rib space segment 702 includes pleural line indicators 706 of a pleural line. The pleural line indicators 706 may be positioned on a bottom edge (e.g., lower boundary) of the pleural line and may visually indicate pixels of the annotated 3D rib space segment 702 that are identified as the pleural line. For example, a pleural line is indicated by the pleural line indicators 706. In other examples, the pleural line indicators 706 may be traced (e.g., with a continuous line) to visually indicate the pleural line in the annotated 3D rib space segment 702. A vertical location of each of the pleural line indicators 706 may be different and may reflect a curvature, protrusion, cavity, and/or other irregularities in the pleural line. For example, a first pleural indicator may have a higher vertical location relative to a second pleural indicator. Ultrasound image data vertically below the pleural line (e.g., below each of the pleural line indicators 706 and spaces therebetween) may be noise and thus may not indicate a topography of the pleural line. For example, a protrusion 718 that extends beyond (e.g., below) the pleural line indicators 706 may be an abnormality of the pleural surface 716 and may be indicative of a pathology. The 3D pleural surface image 704 shows the 3D pleural surface 716, that is generated from the annotated 3D rib space segment 702 by removing dynamic 3D ultrasound image data below the pleural line indicated by pleural line indicators 706, as described with respect to the method 600 of FIG. 6.

FIG. 8 shows a first example dynamic panoramic 3D ultrasound image 800. The dynamic panoramic 3D ultrasound image 800 includes a first dynamic rib space segment 802, a second dynamic rib space segment 804, a third dynamic rib space segment 806, and a fourth dynamic rib space segment 808. Each of the dynamic rib space segments are illustrated as the 3D pleural surface image 704 of FIG. 7, for illustration purposes. It is to be understood that, in reality, each of the dynamic rib space segments may show different topography and structures of the thoracic cavity. The dynamic panoramic 3D ultrasound image 800 further includes rib spaces between each of the dynamic rib space segments. A first rib shadow 812 is formed by a first rib, for example, between the first dynamic rib space segment 802 and the second dynamic rib space segment 804. A second rib shadow 814 is formed by a second rib, for example, between the second dynamic rib space segment 804 and the third dynamic rib space segment 806. A third rib shadow 816 is formed by a third rib, for example, between the third dynamic rib space segment 806 and the fourth dynamic rib space segment 808.

FIG. 9 shows a second example dynamic 3D ultrasound image 900. The second example dynamic 3D ultrasound image 900 may be a perspective view of the first example dynamic panoramic 3D ultrasound image 800 of FIG. 8. Additionally, the dynamic 3D ultrasound image 900 shows exemplary topography of the pleural surface 716 of the fourth dynamic rib space segment 808 and the third dynamic rib space segment 806, with the third rib shadow 816 formed therebetween. The pleural surface 716 of the third dynamic rib space segment 806 includes the protrusion 718.

FIG. 10 shows an example display 1001 including the dynamic 3D ultrasound image 900 of FIG. 9. The display 1001 may be the display device 118 of FIG. 1, for example. The display 1001 includes sliders for adjusting a brightness (e.g., a brightness slider 1010), a color/tint (e.g., hue boxes 1012), and a position (e.g., a coordinate selector 1014) of one or more light sources, as well as a reference axis system 1090. The y-axis may be a vertical axis (e.g., parallel to a gravitational axis), the x-axis may be a lateral axis (e.g., horizontal axis), and the z-axis may be a longitudinal axis, in one example. However, the axes may have other orientations, in other examples.

The methods 500 and 600 optionally include applying shading to the 3D pleural surface when outputting the dynamic 3D ultrasound image for display. The 3D pleural surface may be shaded via at least one virtual light 1020 source positioned as if inside the lung. The shading may highlight a protrusion (e.g., the protrusion 718) extending from the 3D pleural surface towards the inside of the lung. For example, the at least one virtual light source may be positioned at a pre-set location within the inside of the lung, such as at an acute angle with respect to the 3D pleural surface and/or a protrusion therefrom. A position, brightness, color, and/or number of virtual light sources may be adjusted in response to receiving user input.

In this way, a processor may automatically generate a dynamic 3D ultrasound image comprising multiple rib spaces and rib shadows. The methods and systems described herein enable visualization of an entire pleural surface in a single image, where the pleural surface may be captured by an ultrasound probe in multiple individual FOV captures. This may eliminate a diagnostic step of searching for specific views of the pleural surface. In this way, a wide range of ultrasound findings and pathologies may be visualized, including pleural irregularities, pneumothorax, and viral and bacterial infections. As a result, an amount of time the healthcare professional spends reviewing the medical images may be reduced, enabling the healthcare professional to focus on patient care and comfort. Further, by arranging the plurality of rib spaces and rib space segments automatically according to an anatomical order, irregularities may be displayed in an anatomically relevant environment in order to further simplify a diagnostic process.

A technical effect of generating a dynamic 3D ultrasound image is that a processing power used by an imaging system may be reduced due to a reduced number of additional imaging scans as a result of increased accuracy and detail of pleural surface image renderings.

The disclosure also provides support for a method for ultrasound imaging, comprising: acquiring dynamic three-dimensional ultrasound image data, generating a plurality of dynamic three-dimensional rib space segments from the dynamic three-dimensional ultrasound image data, generating a dynamic panoramic three-dimensional ultrasound image depicting the plurality of dynamic three-dimensional rib space segments in an anatomical order, temporally synchronizing the plurality of dynamic three-dimensional rib space segments, and outputting the dynamic panoramic three-dimensional ultrasound image for display. In a first example of the method, generating the plurality of dynamic three-dimensional rib space segments comprises: automatically identifying a plurality of rib shadows in the dynamic three-dimensional ultrasound image data, defining boundaries between each of a plurality of rib spaces using the plurality of rib shadows, and splitting the dynamic three-dimensional ultrasound image data into multiple volumes, based on the boundaries between each of the plurality of rib spaces. In a second example of the method, optionally including the first example, the plurality of rib shadows are detected by tracking motion of an ultrasound probe used to capture the dynamic three-dimensional ultrasound image data during an imaging scan. In a third example of the method, optionally including one or both of the first and second examples, the plurality of rib shadows are detected by intensity and/or brightness of pixels of the dynamic three-dimensional ultrasound image data. In a fourth example of the method, optionally including one or more or each of the first through third examples, depicting the plurality of dynamic three-dimensional rib space segments in the anatomical order includes identifying and exposing a pleural surface of each three-dimensional rib space segment, and ordering the plurality of dynamic three-dimensional rib space segments according to the anatomical order based on the pleural surface. In a fifth example of the method, optionally including one or more or each of the first through fourth examples, temporally synchronizing the plurality of dynamic three-dimensional rib space segments includes applying temporal scaling to expand and/or contract one or more of the plurality of dynamic three-dimensional rib space segments. In a sixth example of the method, optionally including one or more or each of the first through fifth examples, the dynamic three-dimensional ultrasound image data are acquired by sweeping a three-dimensional ultrasound probe across multiple rib spaces while acquiring three-dimensional ultrasound image data. In a seventh example of the method, optionally including one or more or each of the first through sixth examples, the three-dimensional ultrasound probe is swept across multiple rib spaces in a sagittal motion from a bottom of a thoracic cavity to a top of the thoracic cavity, or vice versa.

The disclosure also provides support for a method for ultrasound imaging, comprising: acquiring dynamic three-dimensional ultrasound image data, identifying a plurality of rib spaces of the dynamic three-dimensional ultrasound image data, identifying and exposing a pleural surface in each of the plurality of rib spaces, sequentially aligning the plurality of rib spaces according to an anatomical order using image characteristics of the pleural surface, synchronizing a timing of display of each rib space of the plurality of rib spaces such that a start and an end of display of each rib space occurs simultaneously, and displaying the dynamic three-dimensional ultrasound image data as a dynamic three-dimensional ultrasound image. In a first example of the method, sequentially aligning the plurality of rib spaces includes aligning the plurality of rib spaces based on characteristics of each rib space of the plurality of rib spaces, including characteristics of the pleural surface. In a second example of the method, optionally including the first example, synchronizing the timing of display comprises adjusting a playback speed of display of one or more rib spaces. In a third example of the method, optionally including one or both of the first and second examples, synchronizing the timing of display comprises identifying a respiratory cycle in each of the plurality of rib spaces, and synchronizing the respiratory cycle of the plurality of rib spaces. In a fourth example of the method, optionally including one or more or each of the first through third examples, identifying the plurality of rib spaces in the dynamic three-dimensional ultrasound image data comprises detecting a plurality of rib shadows in the dynamic three-dimensional ultrasound image data, and using the plurality of rib shadows as boundaries between each of the plurality of rib spaces. In a fifth example of the method, optionally including one or more or each of the first through fourth examples, using the plurality of rib shadows as boundaries comprises splitting the dynamic three-dimensional ultrasound image data into multiple volumes that show portions of the dynamic three-dimensional ultrasound image data that each include a single rib space. In a sixth example of the method, optionally including one or more or each of the first through fifth examples, sequentially aligning the plurality of rib spaces includes arranging the plurality of rib spaces according to the anatomical order based on image characteristics of each rib space.

The disclosure also provides support for an ultrasound imaging system, comprising: a display device, an ultrasound probe configured to acquire dynamic three-dimensional ultrasound image data, and a processor in electronic communication with the ultrasound probe and the display device, wherein the processor is configured with computer-readable instructions stored on non-transitory memory that, when executed, cause the processor to: generate a plurality of three-dimensional rib space segments from the dynamic three-dimensional ultrasound image data, generate a dynamic panoramic three-dimensional ultrasound image depicting the plurality of three-dimensional rib space segments in an anatomical order, and display, on the display device, the dynamic panoramic three-dimensional ultrasound image, wherein the plurality of three-dimensional rib space segments takes a same amount of time to play. In a first example of the system, the system further comprises: an ultrasound configured to capture dynamic three-dimensional ultrasound image data as the ultrasound probe is swept over an imaging subject. In a second example of the system, optionally including the first example, the ultrasound probe is a matrix array probe. In a third example of the system, optionally including one or both of the first and second examples, the processor is further configured with instructions in the non-transitory memory that, when executed, cause the processor to: generate the plurality of three-dimensional rib space segments in real-time as the ultrasound probe is swept over a corresponding rib space, and identify and expose a pleural surface of each of the plurality of three-dimensional rib space segments. In a fourth example of the system, optionally including one or more or each of the first through third examples, the processor is further configured with instructions in the non-transitory memory that, when executed, cause the processor to: temporally synchronize a timing of display of each of the plurality of three-dimensional rib space segments such that a start time and an end time of display of each rib space occurs simultaneously.

As used herein, an element or step recited in the singular and preceded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present invention. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used in practice as some jurisdictions require them, but this should not be taken to mean that the features in the dependent claims are mutually exclusive.

1. A method for ultrasound imaging, comprising:
acquiring dynamic three-dimensional ultrasound image data;
generating a plurality of dynamic three-dimensional rib space segments from the dynamic three-dimensional ultrasound image data;
generating a dynamic panoramic three-dimensional ultrasound image depicting the plurality of dynamic three-dimensional rib space segments in an anatomical order;
temporally synchronizing the plurality of dynamic three-dimensional rib space segments; and
outputting the dynamic panoramic three-dimensional ultrasound image for display.

2. The method of claim 1, wherein generating the plurality of dynamic three-dimensional rib space segments comprises:
automatically identifying a plurality of rib shadows in the dynamic three-dimensional ultrasound image data;
defining boundaries between each of a plurality of rib spaces using the plurality of rib shadows; and
splitting the dynamic three-dimensional ultrasound image data into multiple volumes, based on the boundaries between each of the plurality of rib spaces.

3. The method of claim 2, wherein the plurality of rib shadows are detected by tracking motion of an ultrasound probe used to capture the dynamic three-dimensional ultrasound image data during an imaging scan.

4. The method of claim 2, wherein the plurality of rib shadows are detected by intensity and/or brightness of pixels of the dynamic three-dimensional ultrasound image data.

5. The method of claim 1, wherein depicting the plurality of dynamic three-dimensional rib space segments in the anatomical order includes identifying and exposing a pleural surface of each three-dimensional rib space segment, and ordering the plurality of dynamic three-dimensional rib space segments according to the anatomical order based on the pleural surface.

6. The method of claim 1, wherein temporally synchronizing the plurality of dynamic three-dimensional rib space segments includes applying temporal scaling to expand and/or contract one or more of the plurality of dynamic three-dimensional rib space segments.

7. The method of claim 1, wherein the dynamic three-dimensional ultrasound image data are acquired by sweeping a three-dimensional ultrasound probe across multiple rib spaces while acquiring three-dimensional ultrasound image data.

8. The method of claim 7, wherein the three-dimensional ultrasound probe is swept across multiple rib spaces in a sagittal motion from a bottom of a thoracic cavity to a top of the thoracic cavity, or vice versa.

9. A method for ultrasound imaging, comprising:
acquiring dynamic three-dimensional ultrasound image data;
identifying a plurality of rib spaces of the dynamic three-dimensional ultrasound image data;
identifying and exposing a pleural surface in each of the plurality of rib spaces;
sequentially aligning the plurality of rib spaces according to an anatomical order using image characteristics of the pleural surface;
synchronizing a timing of display of each rib space of the plurality of rib spaces such that a start and an end of display of each rib space occurs simultaneously; and
displaying the dynamic three-dimensional ultrasound image data as a dynamic three-dimensional ultrasound image.

10. The method of claim 9, wherein sequentially aligning the plurality of rib spaces includes aligning the plurality of rib spaces based on characteristics of each rib space of the plurality of rib spaces, including characteristics of the pleural surface.

11. The method of claim 9, wherein synchronizing the timing of display comprises adjusting a playback speed of display of one or more rib spaces.

12. The method of claim 9, wherein synchronizing the timing of display comprises identifying a respiratory cycle in each of the plurality of rib spaces, and synchronizing the respiratory cycle of the plurality of rib spaces.

13. The method of claim 9, wherein identifying the plurality of rib spaces in the dynamic three-dimensional ultrasound image data comprises detecting a plurality of rib shadows in the dynamic three-dimensional ultrasound image data, and using the plurality of rib shadows as boundaries between each of the plurality of rib spaces.

14. The method of claim 13, wherein using the plurality of rib shadows as boundaries comprises splitting the dynamic three-dimensional ultrasound image data into multiple volumes that show portions of the dynamic three-dimensional ultrasound image data that each include a single rib space.

15. The method of claim 9, wherein sequentially aligning the plurality of rib spaces includes arranging the plurality of rib spaces according to the anatomical order based on image characteristics of each rib space.

16. An ultrasound imaging system, comprising:
a display device;
an ultrasound probe configured to acquire dynamic three-dimensional ultrasound image data; and
a processor in electronic communication with the ultrasound probe and the display device, wherein the processor is configured with computer-readable instructions stored on non-transitory memory that, when executed, cause the processor to:
generate a plurality of three-dimensional rib space segments from the dynamic three-dimensional ultrasound image data;
generate a dynamic panoramic three-dimensional ultrasound image depicting the plurality of three-dimensional rib space segments in an anatomical order; and
display, on the display device, the dynamic panoramic three-dimensional ultrasound image, wherein the plurality of three-dimensional rib space segments takes a same amount of time to play.

17. The ultrasound imaging system of claim 16, further comprising an ultrasound configured to capture dynamic three-dimensional ultrasound image data as the ultrasound probe is swept over an imaging subject.

18. The ultrasound imaging system of claim 17, wherein the ultrasound probe is a matrix array probe.

19. The ultrasound imaging system of claim 16, wherein the processor is further configured with instructions in the non-transitory memory that, when executed, cause the processor to:
generate the plurality of three-dimensional rib space segments in real-time as the ultrasound probe is swept over a corresponding rib space; and
identify and expose a pleural surface of each of the plurality of three-dimensional rib space segments.

20. The ultrasound imaging system of claim 16, wherein the processor is further configured with instructions in the non-transitory memory that, when executed, cause the processor to:
temporally synchronize a timing of display of each of the plurality of three-dimensional rib space segments such that a start time and an end time of display of each rib space occurs simultaneously.
